(19) United States
(12) Patent Application Publication
(10) Pub. No.: US 2021/0252143 A1
(43) Pub. Date: Aug. 19, 2021

(54) METHODS OF TREATING CANCER USING COMBINATION THERAPY

(71) Applicants: Merck Patent GmbH, Darmstadt (DE); Pfizer Inc., New York, NY (US); Mayo Foundation for Medical Education and Research, Rochester, MN (US); VYRIAD, Rochester, MN (US)

(72) Inventors: Stephen J. Russell, Rochester, MN (US); Alice Bexon, Montclair, NJ (US); Kah-Whye Peng, Rochester, MN (US); Rose Marie Diaz, Rochester, MN (US)

(21) Appl. No.: 17/253,175

(22) PCT Filed: Jun. 21, 2019
(86) PCT No.: PCT/US2019/038480

§ 371 Date: Dec. 17, 2020

Related U.S. Application Data
(60) Provisional application No. 62/688,563, filed on Jun. 22, 2018.

(51) Int. Cl. A61K 39/395; A61K 38/21; A61K 9/00; A61K 38/17; A61K 35/766

(52) U.S. Cl. CPC A61K 39/3955; A61K 38/215; A61K 35/766; A61K 38/1709; A61K 9/0019

(57) ABSTRACT

The present application is directed to therapeutic regimens and methods of treating cancer, with the regimens and methods comprising administering to the subject a programmed death-ligand 1 (PD-L1) inhibitor and a recombinant vesicular stomatitis virus that has been engineered to expresses interferon beta (VSV-IFNβ-NIS).

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 56187-501001WO Sequence Listing_ST25.txt, date created: Apr. 8, 2019; size: 10,991 bytes).

### BACKGROUND OF THE INVENTION

### Field of the Invention

The present application is directed to therapeutic regimens and methods of treating cancer, with the regimens and methods comprising administering to the subject a programmed death-ligand 1 (PD-L1) inhibitor and a recombinant vesicular stomatitis virus (rVSV) that has been engineered to expresses interferon beta and a sodium/iodine symporter (VSV-IFNβ-NIS).

### BACKGROUND OF THE INVENTION

In 2015, an estimated 1,658,370 new cases of cancer were diagnosed and 589,430 cancer deaths occurred in the USA. The five-year relative survival rates for all cancer diagnoses in years 2004-2010 was only 68%. Moreover, some cancers have particularly dim prognosis with 5-year relative survival rates of 7% for pancreatic cancer and less than 20% for liver, lung and esophageal cancers; rates for advanced stage malignancies with distant metastases range from 2% for pancreatic cancer to 55% for thyroid cancer.

Chemotherapy remains the standard treatment option for the majority of patients with metastatic and/or advanced cancer. Unfortunately, for many patients, chemotherapy is not curative and their disease will become refractory to therapy. Patients with refractory, metastatic solid tumors have few treatment options. Once a patient has progressed on standard first-line therapy, second-line options have significant variability in their efficacy depending on cancer type. For some cancers, such as breast cancer, second-line options are numerous and allow reasonable clinical benefit for some time. Other cancers, such as head and neck cancer, lack efficacious second-line options. Patients with rare tumors often have fewer options. As a result, many patients with advanced, incurable cancers will seek out cancer clinical trials with the hope of receiving a novel therapeutic agent that may provide clinical benefit.

Cancer immunotherapy is a rapidly emerging therapeutic class that offers the potential for clinical benefit when chemotherapy becomes ineffective. Over the past decade immune checkpoint inhibitors such as ipilimumab, pembrolizumab, atezolizumab and nivolumab have been approved. These approvals were initially for melanoma, but have more recently expanded to other disease types, and additional agents have recently been approved including avelumab and durvalumab. These agents have stimulated the resurgence of immunotherapies in the clinical pipeline. Numerous agents are in development, including oncolytic viral therapy.

In 2015, the first oncolytic viral therapy, Imlygic (talimogene Laherparepvec), was approved for use in patients with locally advanced melanoma. To further understand their safety and efficacy, oncolytic viruses must be evaluated in patients with refractory, solid tumors. Recently, T-Vec, an oncolytic herpes simplex type 1 virus encoding the granulocyte macrophage colony-stimulating factor, was approved by the FDA for treatment of surgically unresectable melanoma, making it the first in class approved in the USA (Andtbacka 2015). Three other phase III trials studying oncolytic virotherapy are underway: intratumoral administration of oncolytic vaccinia virus encoding GMCSF (Pexa-Vec) for treatment of hepatocellular carcinoma, intravesical adenovirus also encoding GMCSF (CG0070) for treatment of urinary bladder cancer and IV reovirus (Reolysin) treatment for head and neck cancer. Among other oncolytic viral clinical trials, a phase 1 study using intratumoral administration of an oncolytic VSV expressing IFNβ (and not expressing a symporter) for treatment of hepatocellular carcinoma is open and recruiting.

Emerging data suggest that the use of checkpoint inhibitors in conjunction with oncolytic viruses can enhance the anti-tumor immune response through release of neoantigens, leading to durable objective responses in a larger proportion of patients than would be expected with the checkpoint inhibitor alone. While some studies suggest that the combination of checkpoint inhibitors and oncolytic viruses may be useful, to date there has been no study examining a combination therapy composed of a checkpoint inhibitor and an oncolytic virus for metastatic colon cancer in humans.

### SUMMARY OF THE INVENTION

The present application is directed to methods of treating metastatic colon cancer, with the methods comprising administering to the subject a programmed death-ligand 1 (PD-L1) inhibitor and a recombinant vesicular stomatitis virus that has been engineered to expresses interferon beta and a sodium/iodine symporter (VSV-IFNβ-NIS).

The present application is also directed to follow on therapeutic regimens for treating metastatic colon cancer comprising a PD-L1 inhibitor and a recombinant vesicular stomatitis virus that has been engineered to expresses interferon beta and a sodium/iodine symporter (VSV-IFNβ-NIS).

The present application is also directed to kits comprising a PD-L1 inhibitor, a VSV-IFNβ-NIS and a package insert comprising instructions for using the PD-L1 inhibitor and the VSV-IFNβ-NIS to treat or delay progression of metastatic colon cancer in a subject.

### BRIEF DESCRIPTION OF THE DRAWINGS

FIG. 1 depicts sequences of avelumab antibodies. The complementarity determining regions (CDRs) are underlined, and the intervening regions without underlining represent the framework regions. The underlined portions of the heavy chain of avelumab (SEQ ID NO:7), in order, are CRDs 1-3 of the heavy chain, respectively, and are also SEQ ID Nos: 1-3 herein. The underlined portions of the light chain of avelumab (SEQ ID NO:9), in order, are CRDs 1-3 of the light chain, respectively, and are also SEQ ID Nos: 4-6 herein.

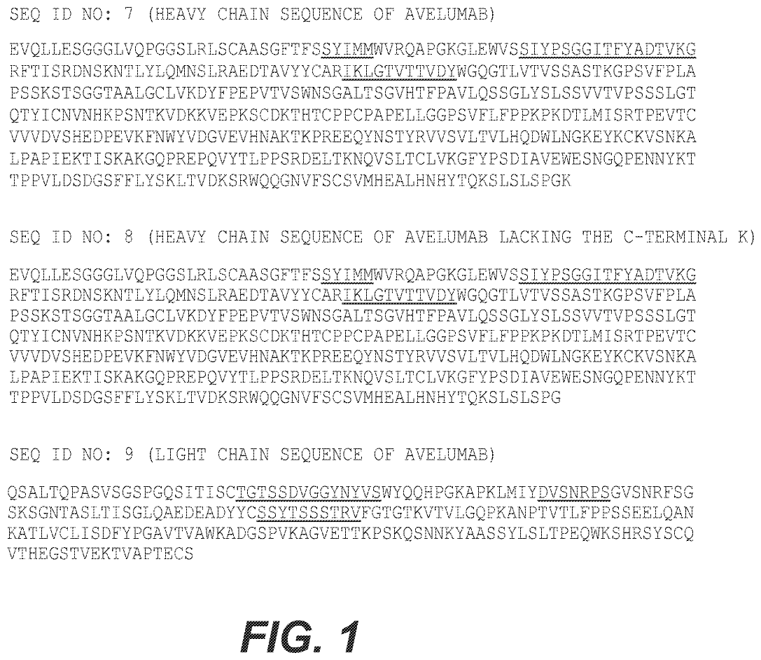

FIG. 2 depicts a study design of a combination therapy for metastatic colon cancer.

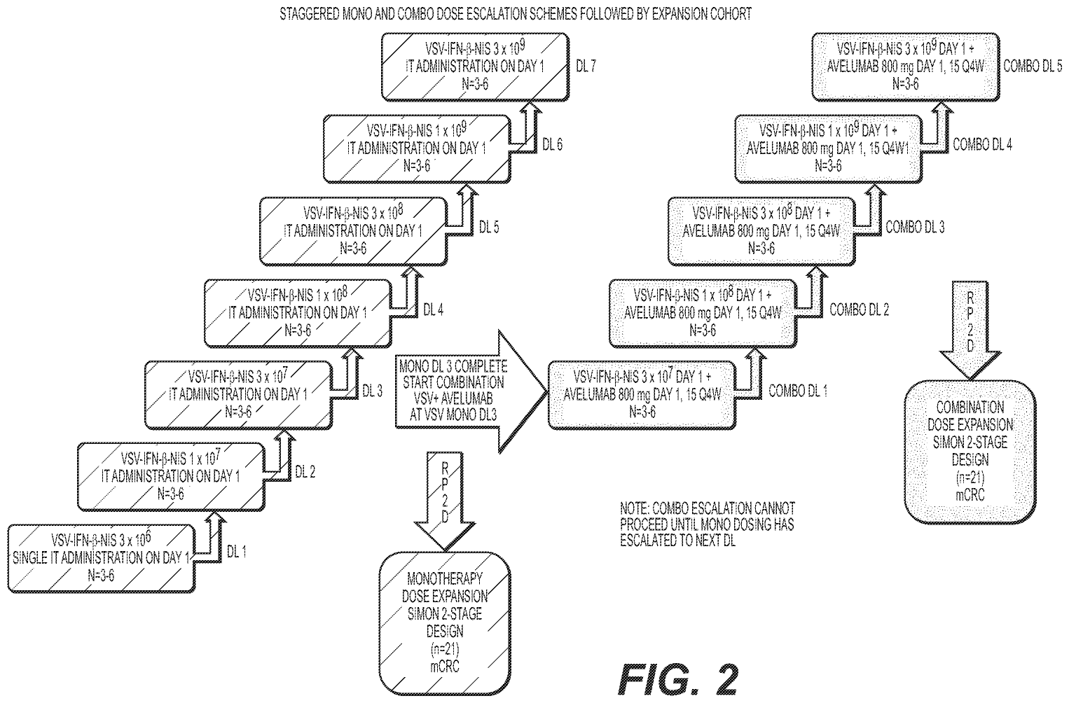

### DETAILED DESCRIPTION OF THE INVENTION

The present application is directed to methods of treating metastatic colon cancer, with the methods comprising administering to the subject a programmed death-ligand 1 (PD-L1) inhibitor and a recombinant vesicular stomatitis virus that has been engineered to expresses interferon beta and a sodium/iodine symporter (VSV-IFNβ-NIS). In the present invention, the terms subject and patient are used interchangeably.

The present application is also directed to follow on therapeutic regimens for treating metastatic colon cancer comprising a PD-L1 inhibitor and a recombinant vesicular stomatitis virus that has been engineered to expresses interferon beta and a sodium/iodine symporter (VSV-IFNβ-NIS).

Human infection with wild type VSV is usually asymptomatic, but can cause an acute, febrile, influenza like illness lasting 3-6 days characterized by fever, chills, nausea, vomiting, headache, retrobulbar pain, myalgia, substernal pain, malaise, pharyngitis, conjunctivitis and lymphadenitis. Complications are generally not seen in humans infected with wild type VSV and fatalities have not been recorded, although a published case of nonfatal meningoencephalitis in a 3-year-old Panamanian child was attributed to VSV infection. A modified Indiana strain VSV has been used in over 17,000 healthy volunteers in an Ebola vaccination program, leading researchers to conclude that the safety profile is considered acceptable in healthy adults. The VSV-based vaccine is generally well tolerated and there have been few vaccine-related adverse events reported. Common adverse events include headache, pyrexia, fatigue, and myalgia, of which the majority are mild to moderate and generally of short duration. Neither shedding of live virus nor human-to-human transmission have been seen.

The vesicular stomatitis virus is a member of the Rhabdoviridae family. The VSV genome is a single molecule of negative-sense RNA that encodes five major polypeptides: a nucleocapsid (N) polypeptide, a phosphoprotein (P) polypeptide, a matrix (M) polypeptide, a glycoprotein (G) polypeptide, and a viral polymerase (L) polypeptide. The nucleic acid sequences of a vesicular stomatitis virus provided herein that encode a VSV N polypeptide, a VSV P polypeptide, a VSV M polypeptide, a VSV G polypeptide and a VSV L polypeptide can be from a VSV Indiana strain as set forth in GenBank Accession Nos. NC_001560 (GI No. 9627229) or can be from a VSV New Jersey strain.

In one embodiment, the methods and regimens of the present invention comprise administration of VSV-IFNβ-NIS. VSV-IFNβ-NIS is a live, virus engineered to express both the human interferon β (hIFN β) gene and the thyroidal sodium iodide symporter (NIS). The virus was constructed by inserting the hIFNβ gene downstream of the M gene and the NIS gene (cDNA) downstream of the gene for the G protein into a full-length infectious molecular clone of an Indiana strain vesicular stomatitis virus (VSV). VSV-IFNβ-NIS is described in PCT/US2011/050227, which is incorporated by reference. The VSV-IFNβ-NIS is a virus comprising an RNA molecule. The RNA encoding the VSV-IFNβ-NIS comprises, or consists essentially of, in a 3′ to 5′ direction, a nucleic acid sequence that is a template for a positive sense transcript encoding a VSV N polypeptide, a nucleic acid sequence that is a template for a positive sense transcript encoding a VSV P polypeptide, a nucleic acid sequence that is a template for a positive sense transcript encoding a VSV M polypeptide, a nucleic acid sequence that is a template for a positive sense transcript encoding a human IFNβ polypeptide, a nucleic acid sequence that is a template for a positive sense transcript encoding a VSV G polypeptide, a nucleic acid sequence that is a template for a positive sense transcript encoding a human NIS polypeptide, and a nucleic acid sequence that is a template for a positive sense transcript encoding a VSV L polypeptide. The virus can express both the IFNβ polypeptide and the NIS polypeptide when the virus infects a mammalian cell. This virus, however, is not considered to be a vaccine.

The nucleic acid sequence encoding the human NIS polypeptide of the VSV-IFNβ-NIS is set forth in GenBank Accession Nos. NM_000453.2 (GI No.164663746), BC105049.1 (GI No. 85397913), or BC105047.1 (GI No. 85397519), all of which are incorporated by reference. The nucleic acid encoding the human IFNβ polypeptide of the VSV-IFNβ-NIS is set forth in GenBank Accession No. NM_002176.2 (GI No. 50593016).

VSV-IFNβ-NIS propagates on BHK cells with similar kinetics to the parental strain of virus and can be grown to high titers. It propagates selectively in human cancer cells since many of them cannot mount an effective antiviral response mediated via the IFN pathway. However, IFN production from infected cells will serve to protect noncancer cells from the effects of the virus. As a result, the virus is directly cytopathic to tumor cells leading to rapid lysis with amplification of the virus. VSV-IFNβ-NIS infected tumor cells also express NIS, a membrane ion channel that actively transports iodide into cells. Radioiodine uptake by cells expressing NIS provides the basis for in vivo imaging with 99mTc pertechnetate or radioiodine I-123 that can reveal the time dependent profile of VSV-IFNβ-NIS gene expression and the location of VSV-IFNβ-NIS infected cells during virus spread and elimination.

Another VSV has been engineered to express only human interferon β (hIFN β) gene without symporter (VSV-IFNβ). This VSV-IFNβ is a different vector altogether and one of skill in the art will appreciate that the differences between VSV-IFNβ and VSV-IFNβ-NIS are more than simply inclusion of a symporter. For example, the placement of the IFNβ transgene is different between VSV-IFNβ-NIS and VSV-IFNβ, and this placement affects virus lifecycle. Indeed, VSV-IFNβ-NIS replicates much slower than VSV-IFNβ, and this difference in replication may directly impact safety and efficacy of the VSV-mediated treatment. Accordingly, the methods and regimens of the present invention do not include the use of VSV-IFNβ, and instead include the use of VSV-IFNβ-NIS.

In certain embodiments of the methods and regimens of the present invention, the PD-L1 inhibitors comprise an antibody or an antigen binding fragment thereof that specifically binds PD-L1. In specific embodiments, the anti-PD-L1 antibody or fragment thereof comprises a heavy chain and a light chain, with the heavy chain comprising three complementarity determining regions (CRDs) having amino acid sequences of SEQ ID NOs: 1, 2 and 3, respectively, and the light chain comprises three CDRs having amino acid sequences of SEQ ID NOs: 4, 5 and 6, respectively.

Heavy Chain CDR Sequences of avelumab
CDR1
(SEQ ID NO: 1)
SYIMM
CDR2
(SEQ ID NO: 2)
SIYPSGGITF YADTVKG
CDR3
(SEQ ID NO: 3)
IKLGTVTTVDY
Light Chain CDR Sequences of avelumab
CDR1
(SEQ ID NO: 4)
TGTSSDVGGYNYVS
CDR2
(SEQ ID NO: 5)
DVSNRPS
CDR3
(SEQ ID NO: 6)
SSYTSSSTRV

In another specific embodiment of the methods and regimens of the present invention the anti-PD-L1 antibody or fragment thereof comprises a heavy chain and a light chain, wherein the heavy chain comprises the amino acid sequence of SEQ ID NOs: 7 or 8 and the light chain comprises an amino acid sequence of SEQ ID NO: 9.

In specific embodiments, the PD-L1 inhibitor is an antibody or fragment thereof that mediates antibody-dependent cell-mediated cytotoxicity (ADCC). In more specific embodiments, the anti-PD-L1 inhibitor is avelumab, which selectively binds to PD-L1 and competitively blocks its interaction with PD-1. Compared with anti-PD-1 antibodies that target T-cells, avelumab targets tumor cells, and therefore may have a lower risk of autoimmune-related safety issues, as blockade of PD-L1 may leave the PD-L2/PD-1 pathway intact to promote peripheral self-tolerance.

Avelumab (BAVENCIO™) is a fully human monoclonal antibody (mAb) of the IgG1 isotype that specifically targets and blocks PD-L1. Avelumab is the International Nonproprietary Name (INN) for the anti-PD-L1 monoclonal antibody MSB0010718C and has been described by its full length heavy and light chain sequences in PCT Publication No. WO 2013/079174, in which it is referred to as A09-246-2, which is hereby incorporated by reference in its entirety. The glycosylation and truncation of the C-terminal lysine in avelumab's heavy chain is also described in PCT Publication No. WO 2017/097407, which is incorporated by reference in its entirety. Avelumab has been in clinical development for the treatment of Merkel Cell Carcinoma (MCC), non-small cell lung cancer (NSCLC), urothelial carcinoma (UC), renal cell carcinoma (RCC) and a number of other cancer conditions.

The term “colon cancer” is used interchangeably with “colorectal cancer” herein. In certain embodiments of the methods and regimens of the present invention, the subject has been diagnosed with colon cancer. The colon cancer can be any stage. In select embodiments, the subject has been diagnosed with stage 0, stage 1, stage 2 (and of stages 2A, 2B, 2C), stage 3 (any of stages 3A, 3B, 3C) or stage 4 (any of stages 4A, 4B) colon cancer. In a specific embodiment, the subject has been diagnosed with metastatic colon cancer. The term “metastatic” includes the cancer spreading into one or more lymph nodes, as well as spreading into one or more organs or tissues. In one specific embodiment, the metastatic colon cancer that is being treated using the methods and regimens disclosed herein is metastatic colon cancer that has spread to one or more organs or tissues that is not near the colon or one or more distant lymph nodes. In additional specific embodiments, the metastatic colon cancer that is being treated using the methods and regimens disclosed herein has spread to at least one organ or tissue selected from lung, kidney, liver, ovary, brain, peritoneum, bone, adrenal gland and muscle.

The term treat (or treatment) is used herein to mean performance of the methods or administration of the regimens described herein to attenuate any symptom such as but not limited to tumor growth or spread, or to prevent additional symptoms from arising. The term “delay the progression” is used to mean that the symptoms, such as tumor growth or spread, do not increase over time.

The methods and regimens of the present invention also include administering the PD-L1 inhibitor and the VSV-IFNβ-NIS to the subject following disease progression after the subject has received at least one, two or three lines of colon cancer therapy. In other words, the methods and regimens are also contemplated to be applied after a first line colon cancer therapy has failed and the disease has progressed. The methods and regimens are also contemplated to be applied after first and second line colon cancer therapies have failed and the disease has progressed. The methods and regimens are also contemplated to be applied after first, second and third line colon cancer therapies have failed and the disease has progressed.

As used herein, the term “disease progression” means that the cancer has continued to grow or spread after the subject has received at least one, two or three lines of colon cancer therapy. Disease progression can also includes instances where the cancer tumors do not shrink or become smaller, i.e., growth is static, after the subject has received at least one, two or three lines of colon cancer therapy. Disease progression can also includes instances where the number of detectable tumors in the subject increases after the subject has received at least one, two or three lines of colon cancer therapy. Disease progression also includes instances where the subject was in remission from a previous line of therapy and the cancer, even a single growth or tumor, has become detectable again. The cancer that is detectable after a period of remission need not necessarily be detectable within the colon or surrounding tissue or lymph nodes, as often times cancer that becomes detectable after a period of remission may return to a different organ or tissue than the colon. The methods and regimens of the present invention include administering the PD-L1 inhibitor and the VSV-IFNβ-NIS to the subject following disease progression after the subject has received at least one, two or three lines of colon cancer therapy, wherein the disease progression is such that the cancer has returned to a different organ or tissue of the body other than the colon and/or the immediately surrounding lymph nodes. Of course, the methods and regimens of the present invention include administering the PD-L1 inhibitor and the VSV-IFNβ-NIS to the subject following disease progression after the subject has received at least one, two or three lines of colon cancer therapy, wherein the disease progression is such that the cancer has returned to the colon and/or the immediately surrounding lymph nodes.

In select embodiments, these first, second or third lines of colon cancer therapies that are administered prior to the application of the methods and regimens described herein comprise administration of at least one therapeutic compound selected from the group consisting of 5-Fluorouracil (5-FU), Capecitabine (XELODA™), Irinotecan (CAMPTOSAR™), Oxaliplatin (ELOXATIN™), Trifluridine, Tipiracil (LONSURF™), Panitumumab (VECTIBIX™), Cetuximab (ERBITUX™), Bevacizumab (AVASTIN™), Ramucirumab (CYRAMZA™), Aflibercept (ZALTRAP™), Regorafenib (STIVARGA™) and Avelumab (BAVENCIO™).

In select embodiments, these first, second or third lines of colon cancer therapies that are administered prior to the application of the methods and regimens described herein comprise radiation treatment.

As used herein, the term “administer” means introducing a therapeutic agent into or on the subject. The routes of administration for each of the VSV-IFNβ-NIS and the PD-L1 inhibitor can be the same or different from one another. The routes of administration for the VSV-IFNβ-NIS and/or the PD-L1 inhibitor include but are not limited to intravenous (IV), intratumoral (IT), intraperitoneal (IP), intramuscular (IM), subcutaneous, oral, nasal or rectal.

In specific embodiments, the PD-L1 inhibitor, such as avelumab, is administered to the subject intravenously (IV) or intraturmorally (IT) or both. In one specific embodiment, the PD-L1 inhibitor, such as avelumab, is administered to the subject intravenously. The total dose of the PD-L1 inhibitor, such as avelumab, can be administered in an amount that at least about 200 mg, 300 mg, 400 mg, 600 mg, 800 mg, 1000 mg, 1200 mg, 1400 mg, 1600 mg, 1800 mg or 2000 mg per administration. Of course the clinician can adjust the concentration of the avelumab, generally speaking in saline solution, in a range of from about 5 mg/kg to about 20 mg/kg. In specific embodiments, the avelumab can be administered to the subject at a concentration of at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg or at least about 20 mg/kg. In more specific embodiments, the avelumab can be administered to the subject intravenously at a concentration of at least about 5 mg/kg, at least about 6 mg/kg, at least about 7 mg/kg, at least about 8 mg/kg, at least about 9 mg/kg, at least about 10 mg/kg, at least about 11 mg/kg, at least about 12 mg/kg, at least about 13 mg/kg, at least about 14 mg/kg, at least about 15 mg/kg, at least about 16 mg/kg, at least about 17 mg/kg, at least about 18 mg/kg, at least about 19 mg/kg or at least about 20 mg/kg.

In one specific embodiment, the PD-L1 inhibitor, such as avelumab, is administered intravenously to the subject via an infusion. Typically, infusions are performed by pump administration or “drip” (using gravity), and the methods and regimens described herein contemplate both pump infusion or drip infusion of the PD-L1 inhibitor, such as avelumab. The methods and regimens described herein also contemplate both pump infusion or drip infusion of the VSV-IFNβ-NIS.

In select embodiments, the methods and regimens described herein comprise administration of the PD-L1 inhibitor, such as avelumab, via an infusion over a period of time. For example, the infusion of the PD-L1 inhibitor, such as avelumab, may last between about 10 minutes and about 180 minutes, between about 20 minutes and about 150 minutes, between about 30 minutes and about 120 minutes, between about 40 minutes and about 90 minutes, between about 50 minutes and about 80 minutes, between about 45 minutes and about 75 minutes and between about 50 minutes and about 70 minutes.

In select embodiments, the methods and regimens described herein comprise administering VSV-IFNβ-NIS is intravenously, intraturmorally or both. In one specific embodiment, the VSV-IFNβ-NIS is administered intratumorally. In more specific embodiments, the VSV-IFNβ-NIS is administered to the subject at a dose of at least about 3×10⁶ 50% tissue culture infective dose (TCID₅₀), 1×10⁷ TCID₅₀, 3×10⁷ TCID₅₀, 1×10⁸ TCID₅₀, 3×10⁸ TCID₅₀, 1×10⁹ TCID₅₀, or 3×10⁹ TCID₅₀. In even more specific embodiments, the VSV-IFNβ-NIS is administered to the subject intratumorally at a dose of at least about 3×10⁶ TCID₅₀, 1×10⁷ TCID₅₀, 3×10⁷ TCID₅₀, 1×10⁸ TCID₅₀, 3×10⁸ TCID₅₀, 1×10⁹ TCID₅₀, or 3×10⁹ TCID₅₀.

In one embodiment of the methods and regimens of the present invention, the VSV-IFNβ-NIS and the PD-L1 inhibitor are administered sequentially. In one specific embodiment, the VSV-IFNβ-NIS is administered the subject prior to the administration of the PD-L1 inhibitor, such as avelumab. In one specific embodiment, the VSV-IFNβ-NIS is administered the subject after the administration of the PD-L1 inhibitor, such as avelumab. When the PD-L1 inhibitor, such as avelumab, is administered after the administration of the VSV-IFNβ-NIS, the PDL-1 inhibitor, such as avelumab, can be administered at least one-half, one, two, three, four, five, six, seven, eight, nine ten, 11, 12, 13, 14, 15, 16, 17, 18, 19, 20, 21, 22, 23, 24, 25, 26 27, 28, 29 or 30 days after the initial administration of the VSV-IFNβ-NIS.

When administered sequentially, the methods and regimens of the present invention include scenarios where administration of both the PD-L1 and the VSV-IFNβ-NIS are under the direction, control or supervision of an attending physician. The methods and regiments of the present invention also include a scenario where the physician has prescribed the VSV-IFNβ-NIS for administration that is not under the direction, control or supervision of an attending physician, such as but not limited to, self-administration, out-patient administration, off-site administration, prior to the first administration of the PD-L1 inhibitor.

In another embodiment, the VSV-IFNβ-NIS and the PD-L1 inhibitor, such as avelumab, are co-administered with one another. As used herein, the term co-administer is used to mean that two or more compounds or agents are administered close in time to one another. While co-administer can include “simultaneous” administration of the two or more compounds, the term co-administer is used herein to include time frames that are not simultaneous but instead take into account small windows of time between the administration of the two or more compounds. For example, the VSV-IFNβ-NIS and the PD-L1 inhibitor, such as avelumab, are considered to be “co-administered” if the two compounds are administered less than 12 hours apart, such as but not limited to, about 15 minutes or less, about 30 minutes or less, about 45 minutes or less, about 60 minutes or less, about 1.5 hours or less, about 2 hours or less, about 2.5 hours or less, about 3 hours or less, about 3.5 hours or less, about 4 hours or less, about 4.5 hours or less, about 5 hours or less, about 5.5 hours or less, about 6 hours or less, about 6.5 hours or less, about 7 hours or less, about 7.5 hours or less, about 8 hours or less, about 8.5 hours or less, about 9 hours or less, about 9.5 hours or less, about 10 hours or less, about 10.5 hours or less, about 11 hours or less, about 11.5 hours or less, or less than about 12 hours. In instances where the administration of the PD-L1 inhibitor, such as avelumab, is administered via an IV drip or infusion, the time window for considering when a compound is co-administered would be the period of time from when the first compound is completely dosed to when administration of the next compound begins.

In additional embodiments, methods and regimens of the present invention include the VSV-IFNβ-NIS being administered more than once to the subject. In one specific embodiment, the VSV-IFNβ-NIS is administered prior to and also after the administration of the PD-L1 inhibitor, such as avelumab. In another specific embodiment, the VSV-IFNβ-NIS is administered more than once after the initial administration of the PD-L1 inhibitor, such as avelumab.

In additional embodiments, methods and regimens of the present invention include administering the PD-L1 inhibitor and/or the VSV-IFNβ-NIS with a pharmaceutically acceptable carrier. The PD-L1 inhibitor and/or the VSV-IFNβ-NIS compositions of the present invention can be prepared by techniques known to those skilled in the art and comprise, for example, an therapeutically effective amount of the PD-L1 inhibitor and/or the VSV-IFNβ-NIS disclosed herein, optionally in combination with or fused to or conjugated to one or more other immunogens, including lipids, phospholipids, carbohydrates, lipopolysaccharides, inactivated or attenuated whole organisms and other proteins, a pharmaceutically acceptable carrier, optionally an appropriate adjuvant, and optionally other materials traditionally used in the art.

The PD-L1 inhibitor and/or the VSV-IFNβ-NIS and the pharmaceutically acceptable carrier may be prepared as an aqueous solution, emulsion or suspension or may be a dried preparation. Such preparations are referred to as the pharmaceutical preparations of the present invention hereafter. Appropriate carriers are known to those skilled in the art and include stabilizers, diluents, and buffers. Suitable stabilizers include carbohydrates, such as sorbitol, lactose, mannitol, starch, sucrose, dextran, and glucose, and proteins, such as albumin or casein. Suitable diluents include saline, Hanks Balanced Salts, and Ringers solution. Suitable buffers include an alkali metal phosphate, an alkali metal carbonate, or an alkaline earth metal carbonate. In select embodiments, the composition of the invention is formulated for administration to humans.

The pharmaceutical preparations of the present invention can be prepared by techniques known to those skilled in the art, given the teachings contained herein. Generally, a therapeutic agent, such as the PD-L1 inhibitor and/or the VSV-IFNβ-NIS, is mixed with the carrier to form a solution, suspension, or emulsion. One or more of the additives discussed herein may be added in the carrier or may be added subsequently. The preparations disclosed here may be desiccated or lyophilized, for example, by freeze drying or spray drying for storage or formulations purposes. They may be subsequently reconstituted into liquid vaccines by the addition of an appropriate liquid carrier or administered in dry formulation using methods known to those skilled in the art, particularly in capsules or tablet forms.

An effective amount of the pharmaceutical preparation of the invention should be administered, in which “effective amount” is defined as an amount that is sufficient to produce a desired prophylactic, therapeutic or ameliorative response in a subject, including but not limited to a response indicating that the virus is effectively propagating. The amount needed will vary depending upon the therapeutic agent used and the species and weight of the subject to be administered, but may be ascertained using standard techniques.

The pharmaceutical preparations of the present invention may further contain one or more auxiliary substance, such as wetting or emulsifying agents, pH buffering agents, or adjuvants to enhance the effectiveness thereof. Each component of the dosing regimen may be administered to subjects by a variety of administration routes, including parenterally, intradermally, intraperitoneally, subcutaneously or intramuscularly. The route of administration of each component of the dosing regimen, e.g., the PD-L1 inhibitor and the VSV-IFNβ-NIS, need not be the same.

The PD-L1 inhibitor and/or the VSV-IFNβ-NIS compositions may be formulated and delivered in a manner to evoke an appropriate response at mucosal surfaces. Thus, the PD-L1 inhibitor and the VSV-IFNβ-NIS compositions may be administered to mucosal surfaces by, for example, the nasal, oral (intragastric), or intrarectal routes. Other modes of administration include but are not limited to oral formulations. Oral formulations may include normally employed incipients such as, for example, pharmaceutical grades of saccharine, cellulose and magnesium carbonate. These compositions can take the form of microspheres, solutions, suspensions, tablets, pills, capsules, sustained release formulations or powders. The PD-L1 inhibitor and/or the VSV-IFNβ-NIS compositions can be administered in a manner compatible with the dosage formulation, and in such amount as will be therapeutically effective, protective or immunogenic. The PD-L1 inhibitor and/or the VSV-IFNβ-NIS compositions may optionally comprise an adjuvant.

In some embodiments, the PD-L1 inhibitor and/or the VSV-IFNβ-NIS compositions may be used in combination with or conjugated to one or more targeting molecules for delivery to specific cells of the subject. Some examples include but are not limited to vitamin B12, bacterial toxins or fragments thereof, monoclonal antibodies and other specific targeting lipids, proteins, nucleic acids or carbohydrates.

In other embodiments, the PD-L1 inhibitor and/or the VSV-IFNβ-NIS compositions of the present invention can be administered with sterile saline or sterile buffered saline colloidal dispersion systems, such as macromolecule complexes, nanocapsules, silica microparticles, tungsten microparticles, gold microparticles, microspheres, beads and lipid based systems including oil-in-water emulsions, micelles, mixed micelles and liposomes. One type of colloidal system for use as a delivery vehicle in vitro and in vivo is a liposome (i.e., an artificial vesicle). The uptake of naked nucleic acid molecules (such as the nucleic acid encoding the VSV-IFNβ-NIS) may be increased by incorporating the nucleic acid molecules into and/or onto biodegradable beads, which are efficiently transported into the cells. The preparation and use of such systems is well known in the art.

In other embodiments, nucleic acid molecules (such as the nucleic acid encoding the VSV-IFNβ-NIS) can be associated with agents that assist in cellular uptake. It can be formulated with a chemical agent that modifies the cellular permeability, such as bupivacaine (see, e.g., WO 94/16737). Cationic lipids are also known in the art and are commonly used for nucleic acid molecules delivery. Such lipids include LIPOFECTIN™, also known as DOTMA (N-[1-(2,3-dioleyloxy)propyl]-N,N,N-trimethylammonium chloride), DOTAP (1,2-bis(oleyloxy)-3-(trimethylammonio)propane, DDAB (dimethyldioctadecylammonium bromide), DOGS (dioctadecylamidologlycy spermine) and cholesterol derivatives such as DC-Chol (3 beta-(N-(N′,N′-dimethyl aminomethane)-carbamoyl) cholesterol. A description of these cationic lipids can be found in EP 187,702, WO 90/11092, U.S. Pat. No. 5,283,185, WO 91/15501, WO 95/26356, and U.S. Pat. No. 5,527,928. Cationic lipids for nucleic acid molecules delivery can be used in association with a neutral lipid such as DOPE (dioleyl phosphatidylethanolamine) as described in, e.g., WO 90/11092. Other transfection facilitation compounds can be added to a formulation containing cationic liposomes. They include, e.g., spermine derivatives useful for facilitating the transport of nucleic acid molecules through the nuclear membrane (see, for example, WO 93/18759) and membrane-permeabilizing compounds such as GAL4, Gramicidine S and cationic bile salts (see, for example, WO 93/19768).

In additional embodiments, methods and regimens of the present invention include the PD-L1 inhibitor, such as avelumab, being administered more than once to the subject. In one specific embodiment, the PD-L1 inhibitor, such as avelumab, is administered prior to and also after the administration of the VSV-IFNβ-NIS. In another specific embodiment, the PD-L1 inhibitor, such as avelumab, is administered more than once after the initial administration of the VSV-IFNβ-NIS.

In instances where multiple doses of PD-L1 inhibitor, such as avelumab, are administered to the subject, the second and subsequent dose can be administered at least about one, two, three, four, five, six, seven, eight, nine ten, 11, 12, 13, 14, 15, 16, 17, 18, 19, 20, 21, 22, 23, 24, 25, 26 27, 28, 29 or 30 days after the initial or previous administration of the PD-L1 inhibitor, such as avelumab.

The multiple doses of PD-L1 inhibitor, such as avelumab, can occur over a predetermined or prescribed period of time. For example, the PD-L1 inhibitor, such as avelumab, may be administered once a week for multiple weeks. In other example, the PD-L1 inhibitor, such as avelumab, may be administered more than once a week, e.g., two, three, four, five, six or seven times a week, for multiple weeks. In specific embodiments, the PD-L1 inhibitor, such as avelumab, is administered twice a week for at least one, two, three, four, five, six, seven, eight, nine, 10,11, 12, 13, 14, 15, 16, 17, 18, 19 or 20 weeks. In one specific embodiment, the PD-L1 inhibitor, such as avelumab, is administered twice a week, at a dose of about 10 mg/kg (for each administration) for at least one, two, three, four, five, six, seven, eight, nine, 10 ,11, 12, 13, 14, 15, 16, 17, 18, 19 or 20 weeks.

In instances where multiple doses of VSV-IFNβ-NIS are administered to the subject, the second and subsequent dose can be administered at least about one, two, three, four, five, six, seven, eight, nine ten, 11, 12, 13, 14, 15, 16, 17, 18, 19, 20, 21, 22, 23, 24, 25, 26 27, 28, 29 or 30 days after the initial or previous administration of the VSV-IFNβ-NIS.

The multiple doses of VSV-IFNβ-NIS can occur over a predetermined or prescribed period of time. For example, the VSV-IFNβ-NIS may be administered once a week for multiple weeks. In other example, the VSV-IFNβ-NIS may be administered more than once a week, e.g., two, three, four, five, six or seven times a week, for multiple weeks. In specific embodiments, the VSV-IFNβ-NIS is administered twice a week for at least one, two, three, four, five, six, seven, eight, nine, 10, 11, 12, 13, 14, 15, 16, 17, 18, 19 or 20 weeks. In one specific embodiment, the VSV-IFNβ-NIS is administered twice a week, at a dose of at least about 3×10⁶ TCID₅₀, (for each administration) for at least one, two, three, four, five, six, seven, eight, nine, 10, 11, 12, 13, 14, 15, 16, 17, 18, 19 or 20 weeks.

In additional embodiments, the methods and regimens further comprise administering an antihistamine and/or paracetamol to the subject prior to administration of the PD-L1 inhibitor, such as avelumab. If the methods and regimens include multiple doses of the PD-L1 inhibitor, such as avelumab, then each administration of the PD-L1 inhibitor may or may not include a pre-administration of antihistamine and/or paracetamol.

The invention is also directed to kits, wherein the kits comprise at least two containers with the first container containing at least one dose of VSV-IFNβ-NIS and the second container containing at least one dose of a PD-L1 inhibitor, such as avelumab.

### EXAMPLES

### Example 1

### Generation of VSV-IFNβ-NIS

Briefly, nucleic acid sequences of desired transgenes were generated with specific restriction sites using PCR. The transgenes were inserted at specific insertion sites into a plasmid encoding the positive strand of the VSV genome in a 5′ to 3′ orientation. The modified plasmid was expanded and infective virus was recovered by infection with vaccinia virus coding for required T7 polymerase and transfection of VSV viral proteins N, P and L. This allowed production of required viral polypeptides allowing generation of the negative sense viral genome that was assembled into infective virions. Recovered virus was amplified, and infective dose was measured on an appropriate cell line in culture, e.g., BHK-21 cells. The nucleic acid sequence of the human IFN beta polypeptide used to make these vesicular stomatitis viruses is set forth in GenBank Accession No. NM_002176.2 (GI No.50593016). The nucleic acid sequence of the human NIS polypeptide used to make these vesicular stomatitis viruses is set forth in GenBank Accession No. NM_000453.2 (GI No.164663746).

When nucleic acid encoding the human NIS polypeptide was inserted upstream of the nucleic acid encoding the VSV G polypeptide, functional virions were not generated because the NIS expression levels appear to have been too high for cells to remain viable and allow viral propagation. Inserting nucleic acid encoding the NIS polypeptide downstream of the nucleic acid encoding the VSV G polypeptide resulted in the generation of functional NIS-expressing virions due to lower quantities of NIS polypeptide being produced thereby allowing not only efficient viral propagation, but also sufficient quantities of NIS polypeptide for functional iodide uptake in infected cells.

Inserting a nucleic acid encoding an IFNβ polypeptide between the nucleic acid encoding the VSV M polypeptide and the nucleic acid encoding the VSV G polypeptide resulted in viruses that infected cells and produced a significantly increased level of IFNβ polypeptide expression that was observed in the supernatant from infected cells. The VSV-IFNβ-NIS viruses were able to replicate efficiently in vitro in infected cells and also express high levels of functional NIS.

### Example 2

### Study Design

This is a two-part open label phase I study designed to determine the safety profile, maximum tolerate dose (MTD), pharmacokinetics (PK) and tumor and biomarker response after intratumoral (IT) administration of a single dose of VSV-IFNβ-NIS, with intravenous (IV) avelumab every two weeks, in patients with refractory advanced/metastatic solid tumors. The study consists of a combination therapy with avelumab in patients with refractory advanced/metastatic solid tumors followed by a monotherapy and combination therapy dose expansion at the VSV-IFNβ-NIS MTD or RP2D in patients with metastatic colon cancer.

For dose escalation, patients are required to have at least 1 measurable lesion per RECIST 1.1 amenable for a one-time IT injection of VSV-IFNβ-NIS. At least one patient per cohort is required to have at least 2 measurable lesions per RECIST 1.1, one for a one-time IT injection of VSV-IFNβ-NIS and one to be used as a control. Priority enrollment will be granted to patients with 2 measurable lesions per RECIST 1.1. At least one patient per dose level should have metastatic colon cancer. To fulfil these requirements, 3 or 4 patients will be required per dose cohort.

Maximum tolerated dose (MTD) will be defined and determined by using a modified Fibonacci cohort 3+3 design. Treatment will start at dose level 1 (DL1). Dose escalation to DL2 and subsequent doses will not occur until the patients in the current dose level have been observed for at least 3 weeks (day 21). If no dose-limiting toxicities (DLTs) have occurred by that time point, in those 3 participants, dose escalation to the next level can occur. At any time, if 1 of 3 participants experiences a DLT during this 3-week time period in a dose level, an additional 3 patients must be enrolled in that dose level. The first 3 participants in each cohort must be enrolled one at a time with at least a 7-day window between treatments of the participant to enrollment of another. If only 1 out of the 6 experience a DLT after 3 weeks of observation at that dose level, escalation to the next dose level with 3 participants can occur as previously described. If a DLT occurs in 2 of 6 of the participants of a dose level, it will be determined that the MTD has been exceeded. In that case, the prior dose level will be expanded, if not already done so, to a total of 6 patients to confirm that only 1 of 6 have had a DLT. This would then be the final recommended phase 2 dose (RP2D).

In the case that 2 or more patients experience DLT at the starting dose DL1, then the study will be amended or terminated. Once monotherapy dose level 3 has been explored with VSV-IFNβ-NIS and escalation has proceeded to dose level 4, dose escalation with the combination of VSV-IFNβ-NIS and avelumab will begin. Combination escalation will begin at VSV-IFNβ-NIS dose level 3 and may not proceed to higher escalation doses until the monotherapy escalation has completed for a specific dose level.

If a suspected DLT occurs, a Data Safety Monitoring Board (DSMB) meeting will be held as rapidly as possible. In the meantime, dosing of the ongoing patients in that cohort will continue unless there is reason to suspect an unacceptable safety risk based on the nature and/or severity of the observed DLT. If an individual patient experiences DLT, that patient will discontinue study drugs.

Once the MTD and/or a RP2D is determined in dose escalation, two, two-stage expansion cohorts (one for monotherapy and one for combination therapy) will be opened and both will enroll patients with metastatic colorectal cancer to further characterize the safety, tolerability, pharmacokinetics (PK), pharmacodynamics (PD) and anti-tumor activity of VSV-IFNβ-NIS, with and without avelumab. A Simon's two-stage design will be used for the expansion cohorts. In expansion study stage 1, 12 patients will be accrued and evaluated for efficacy. If there are 0 responses in these 12 patients, the cohort will be stopped. Otherwise, nine additional patients will be accrued and evaluated per the study schedule for a total of 21 patients. The study will not be stopped between stage 1 and stage 2. At least six patients per cohort will be required to have 2 measurable lesions per RECIST 1.1, one for IT injection of VSV-IFNβ-NIS and one to be used as a control. At least six patients per cohort will also be required to undergo SPECT/CT imaging.

Patients in the expansion cohorts will be treated and monitored per the same study procedures as outlined for the dose escalation part of the study.

### Example 3

### Dosing

Trial treatment with VSV-IFNβ-NIS will be given by appropriately trained surgical and interventional radiology sub-investigators. VSV-IFNβ-NIS will be administered IT in one single tumor location as agreed upon by the principal investigator and administering sub-investigator using TB syringes (or equivalent) and 20- to 23-gauge needles, or a Quadrafuse® multiprongneedle for larger tumors. Administration into sites including skin, soft tissue, nodal, lung or liver lesions are permitted. Criteria for administration are as follows:

TABLE 1
Injection Site Size and Image Guidance Criteria

| | Size criteria for | Image guidance |
|---|---|---|
| Injection site | injection¹ | required |
| Skin | ≥1 cm | Photographs pre-injection² |
| Soft tissue (not | ≥1 cm | Ultrasound or CT |
| penetrating skin | | |
| surface and not nodal) | | |
| Nodal | ≥1.5 cm³ | Ultrasound or CT |
| Lung | ≥2 cm | CT |
| Liver (Group B only) | ≥1 cm and ≤4 cm | CT |

¹Measured in the longest dimension
²Photograph should include measuring tape documenting longest measurable dimension
³Nodal measured 1.5 cm in the shortest dimension

Once the VSV-IFNβ-NIS dose for the participant has been assigned, the virus will be thawed and prepared per pharmacy manual protocol and diluted in albumin immediately prior to administration (ideally around 30 minutes but up to 6 hours maximum). The final volume is dependent on the tumor lesion size and is estimated based on the formula

Volume of injection (Vi)=(a\hat{ }2)(b)(0.5)
[a=the shorter diameter and b=the longer diameter] of injectable product.

The volume delivered to the tumor will be dependent on the size of the tumor nodule(s) and will be determined according to the following algorithm:
up to 0.5 mL for tumors of 1.0 to 1.5 cm longest dimension.
up to 1.0 mL for tumors of 1.5 to 2.5 cm longest dimension.
up to 2.0 mL for tumors of 2.5 to 5 cm longest dimension.
up to 4.0 mL for tumors>5 cm longest diameter

Note that for DL 7 only (3×10⁹ TCID₅₀) patients with maximum lesion diameter<1.5 cm cannot be included due to volume constraints.

Once the trial treatment is thawed and diluted, administration will occur as follows:

The injection site will be cleaned with the use of an appropriate agent, such as chlorhexadine or betadine.

The area will be draped in a sterile fashion.

If felt to be necessary by the administering sub-investigator, the study participant will be given minimal sedation per routine standard of care.

Local anesthetic using 1% lidocaine may be used at the injection site if deemed necessary by the administering sub-investigator per routine standard of care. However, this should not be injected directly into the lesion. Adequate analgesia should be accomplished if the local anesthetic is injected around the lesion.

Using appropriate image guidance as outlined in the table below, the administering sub-investigator or designee will draw up the study drug solution from the vial provided by the pharmacy into TB syringes (or equivalent) and administer the VSV-IFNβ-NIS via 20- to 23-gauge needles, or a Quadrafuse® multiprong needle for larger tumors, into the pre-identified tumor site.

For visible lesions, the injection site may be pre-treated with a topical anesthetic agent. VSV-IFNβ-NIS should be injected along multiple different tracks within the lesion in order to obtain as wide a dispersion as possible.

Injection of the viral product should occur slowly. If the tumor site is >2 cm in diameter, this may require multiple injections due to the amount of dissolved viral product. If multiple injections are required, these should be administered approximately 2 cm apart from the prior injection. Total time for the procedure is anticipated to take 30-60 minutes.

Post-procedure dressings should be applied per standard practice. The injection site will be swabbed with alcohol and covered with a dry occlusive dressing (an absorbent pad and an occlusive cover—e.g. Tegaderm® or Tegaderm® with absorbent pad).

Monitoring post-procedure should occur in the treating department or in the Post Anesthesia Care Unit (PACU) for about 2 hours after the procedure with vital signs checked every 15 minutes.

After post-procedure monitoring has ended, study participants will be admitted to the hospital ward in outpatient bed status for monitoring for up to 23 hours after the procedure. Vital signs will be recorded every hour for the first 4 hours on the ward, then every 4 hours for the remainder of the 23 hour stay. Supportive care will be given as outlined in Section 9.4.

Participants will be discharged from monitoring once the 23 hours are done and after being cleared by the principal investigator or sub-investigator. Additional monitoring may be required based on individual toxicities.

Avelumab drug product is a sterile solution intended for IV infusion, as a clear, colorless concentrate for solution presented at concentration of 20 mg/mL in European Pharmacopeia (Ph. Eur.) and United States Pharmacopeia (USP) type I glass vials closed with a rubber stopper and sealed with an aluminum Flip Off® crimp seal closure.

Avelumab will be administered as an IV infusion at a dose of 800 mg over a duration of 1 hour (−10 minutes/+20 minutes) once every 2 weeks. Avelumab will be administered after VSV on day 1.

Avelumab drug product must be diluted with 0.9% saline solution (sodium chloride injection) supplied in an infusion bag; alternatively, a 0.45% saline solution can be used if needed. Refer to the approved US Prescribing Information for dosage preparation instructions.

Mandatory premedication with antihistamine and paracetamol (acetaminophen) for the first 4 doses is requested in all subjects to be treated with avelumab. To mitigate infusion-related reactions, premedication with an antihistamine and with paracetamol (acetaminophen) (for example, 25 to 50 mg diphenhydramine and 500 to 650 mg paracetamol [acetaminophen] iv or oral equivalent) approximately 30 to 60 minutes prior to the first 4 doses of avelumab is mandatory. Premedication should be administered for subsequent avelumab doses based upon clinical judgment and presence/severity of prior infusion reactions. This regimen may be modified based on local treatment standards and guidelines as appropriate. However, the prophylactic use of systemic corticosteroids is not permitted.

As a routine precaution, subjects enrolled in this trial must be observed for 1 hour post infusion for the first 4 infusions, in an area with resuscitation equipment and emergency agents. At all times during avelumab treatment, immediate emergency treatment of an infusion-related reaction or a severe hypersensitivity reaction according to institutional standards must be assured. In order to treat possible hypersensitivity reactions, for instance, dexamethasone 10 mg and epinephrine in a 1:1000 dilution or equivalents should always be available along with equipment for assisted ventilation.

Investigators should also monitor subjects closely for potential immune-related AEs (irAEs), which may become manifest at any time during treatment. Such events include but are not limited to pneumonitis, hepatitis, colitis, endocrinopathies (hypothyroidism, hyperthyroidism, adrenal insufficiency, type 1 diabetes mellitus), myocarditis, myositis, rash. See Section 9.1.10.2 for details on the management of irAEs.

### Example 4

### Assessment of Efficacy

RECIST version 1.1 will be used in this study for assessment of tumor response. While either CT or MRI may be utilized as per RECIST 1.1, CT is the preferred imaging technique in this study.

Tumor measurements for efficacy evaluation will be done 6 weeks (43-day visit) after IT injection of the study drug. A radiological assessment of CR or PR requires confirmatory imaging at least 4 weeks after the initial assessment of response was observed.

Measurable Disease: Tumor lesions: Must be accurately measured in at least one dimension (longest diameter in the plane of measurement is to be recorded) with a minimum size of:
10 mm by CT by computerized tomography (CT scan slice thickness no greater than 5 mm).
10 mm caliper measurement by clinical exam (lesions that cannot be accurately measured with calipers should be recorded as non-measurable).
20 mm by chest x-ray.
Skin lesions: Documentation by color photography, including a ruler to estimate the size of the lesion, is recommended.
Malignant lymph nodes: To be considered pathologically enlarged and measurable, a lymph node must be ≥15 mm in short axis when assessed by CT scan. At baseline and in follow-up, only the short axis will be measured and followed.

Non-Measurable Disease: All other lesions, including small lesions (longest diameter<10 mm or pathological lymph nodes with ≥10 to <15 mm short axis) as well as truly non-measurable lesions. Lesions considered truly non-measurable include: leptomeningeal disease, ascites, pleural or pericardial effusion, inflammatory breast disease, and lymphangitic involvement of skin or lung, abdominal masses, abdominal organomegaly identified by physical exam that is not measurable by reproducible imaging techniques

Target Lesions: The most reproducible measurable lesions, up to a maximum of 2 lesions per organ and 5 lesions in total, representative of all involved organs should be identified as target lesions and recorded and measured at baseline.

Target lesions should be selected on the basis of their size (lesions with the longest diameter), should be representative of all involved organs, and, in addition, should be those that lend themselves to reproducible repeated measurements. Pathological nodes which are defined as measurable and that may be identified as target lesions must meet the criterion of a short axis of 15mm. All target lesions will be calculated and reported as the baseline sum diameters. If lymph nodes are to be included in the sum, then as noted above, only the short axis is added into the sum. The baseline sum diameters will be used as reference to further characterize any objective tumor response.

Non-Target Lesions: All other lesions (or sites of disease) are identified as non-target lesions (chosen based on their representativeness of involved organs and the ability to be reproduced in repeated measurements) and should be recorded at baseline. Measurements of these lesions are not required, but the presence or absence of each should be noted throughout follow-up. Lymph nodes with short axis≥10 mm but <15 mm should be considered non-target lesions. Nodes that have a short axis<10 mm are considered non-pathological and are not recorded or followed.

Evaluation of Target Lesions

Complete Response (CR): Disappearance of all target lesions. Any pathological lymph nodes (whether target or non-target) must have reduction in short axis to <10 mm. Tumor marker results must have normalized.

Partial Response (PR): At least a 30% decrease in the sum of diameters of target lesions, taking as reference the baseline sum diameters.

Stable Disease (SD): Neither sufficient shrinkage to qualify for PR nor sufficient increase to qualify for PD, taking as reference the smallest (nadir) sum of diameters since the treatment started.

Progressive Disease (PD): At least a 20% increase in the sum of the diameters of target lesions, taking as reference the smallest (nadir) sum since the treatment started, or the appearance of one or more new lesions. Requires not only 20% increase, but absolute increase of a minimum of 5 mm over sum.

Evaluation of Non-Target Lesions

Complete Response (CR): Disappearance of all non-target lesions and normalization of tumor markers. All lymph nodes must be non-pathological in size (<10 mm short axis).

Stable Disease (SD): Persistence of one or more non-target lesions and/or persistence of tumor marker level above the normal limits.

Progressive Disease (PD): Appearance of one or more new lesions and/or unequivocal progression of existing non-target lesions. When the patient also has measurable disease there must be an overall level of substantial worsening in non-target disease such that, even in presence of SD or PR in the target disease, the overall tumor burden has increased sufficiently to merit discontinuation of therapy.

TABLE 2
Evaluation of Best Overall Response

| Target Lesions | Non-Target Lesions | New Lesions | Overall response |
|---|---|---|---|
| CR | CR | No | CR |
| CR | Non-CR/non-PD | No | PR |
| CR | NE | No | PR |
| PR | Non-PD or NE | No | PR |
| SD | Non-PD or NE | No | SD |
| Not all | Non-PD | No | NE |
| evaluated | | | |
| PD | Any | Yes or No | PD |
| Any | PD | Yes or No | PD |
| Any | Any | Yes | PD |

CR: disappearance of all target and non-target lesions, normalization of tumor marker level, and reduction in pathological lymph nodes short axis to <10 mm.
PR: 30 % decrease in the sum of diameters of the target lesions compared to baseline.
PD: 20 % increase in the sum of diameters of the target lesions compared to nadir.

When nodal disease is included in the sum of target lesions, and the nodes decrease to “normal” size (<10 mm), they may still have a measurement reported on scans. This measurement should be recorded even though the nodes are normal in order not to overstate progression, should it be based on increase in size of the nodes. As noted earlier, this means that patients with CR may not have a total sum of ‘zero’ on the case report form (CRF).

If there is suspicion of disease progression based on clinical or laboratory findings before the scheduled assessment, an unscheduled assessment should be performed. Patients with a global deterioration of health status requiring discontinuation of treatment without objective evidence of disease progression at that time should be reported as “symptomatic deterioration”. Every effort should be made to document objective progression even after discontinuation of treatment.

### Example 5

### Statistical Considerations

Safety Population: The safety population consists of all patients who receive at least one dose of the study medication. All safety and tolerability evaluations will be based on this analysis set.

PK/PD Population: The PK/PD population includes all patients without protocol deviations affecting interpretability of PK and/or PD.

Efficacy Population: primary efficacy analyses will utilize the treated population, which is identical to the Safety Population.

All analyses will be performed by dose and overall (i.e., overall dose levels) within the pre-specified analysis set (monotherapy and combination therapy will be analyzed separately). Expansion cohorts will be analyzed by therapy (monotherapy-arm/combotherapy-arm).

The number and percentage of patients screened, enrolled, the primary reasons for screening failure, and the primary reason for discontinuation will be displayed. Demographic variables, baseline characteristics, primary and secondary diagnoses, and prior and concomitant therapies will be summarized by treatment either by descriptive statistics or categorical tables.

VSV-IFNβ-NIS and Avelumab exposure data will be reported as total dose administered per patient and relative dose intensity (actual dose/planned dose) summarized by dose cohort and overall.

Concomitant medications will be coded using the current WHO Drug Dictionary and the data will be summarized and presented in tables and listings.

Due to the exploratory nature of these endpoints only descriptive statistics will be used. Results will be presented by dose cohort and overall.

Overall Response Rate (ORR): proportion of patients in the analysis population who have complete response (CR) or partial response (PR) based on RECIST 1.1 imaging on day 43. Frequency and relative frequency will be computed for each (overall, by dose level, and by disease type).

Disease Control Rate (DCR): proportion of patients in the respective analysis population who have complete response (CR), partial response (PR) or stable disease (SD) based on RECIST 1.1 imaging on day 43. Frequency and relative frequency will be computed for each (overall, by dose level, and by disease type).

Tumor response rate of the injected lesion (TNi) and distant lesion (TNd): proportion of patients in the analysis population who have complete response (CR) or partial response (PR) based on RECIST 1.1 imaging on day 43 of TNi and TNd. Frequency and relative frequency will be computed for each (overall, by dose level, and by disease type).

Progression-Free Survival (PFS): time from Day 1 treatment administration to the first documented disease progression or death, whichever occurs first.

Duration of Response (DOR): time from first observation of response to the first documented disease progression or death, whichever occurs first.

Overall Survival (OS): time from Day 1 treatment administration to death due to any cause.

Tumor Necrosis (TN): Injected lesion (TNi) and distant lesion (TNd) necrosis rate will be defined as TNi and TNd≥30% increase in necrosis from baseline, respectively. Frequency and relative frequency will be computed for each (overall, by dose level, and by disease type).

TABLE 3
Statistical Methods for Key Efficacy Endpoints

| Endpoint | Statistical Method | Missing data Approach |
|---|---|---|
| Objective Response | Exact binomial method: | Patients with missing |
| Rate (CR + PR) | Clopper-Pearson 95% | data are considered |
| | confidence interval | non-responders |
| PFS investigator | Summary statistics | Censored at last |
| assessment | using Kaplan-Meier | assessment date or |
| | method | start of new systemic |
| | | therapy |
| DOR | Summary statistics | Censored at last |
| | using Kaplan-Meier | assessment date or |
| | method | start of new systemic |
| | | therapy |
| DCR | Exact binomial | Patients with missing |
| | method: Clopper- | data are considered |
| | Pearson 95% | non-responders |
| | confidence interval | |
| OS | Summary statistics | Censored at last |
| | using Kaplan-Meier | assessment date |
| | method | |
| TN | Frequency and | Patients with missing |
| | relative frequency | data are considered |
| | | non-responders |

The primary safety objective of this trial is to characterize the safety of VSV-IFNβ-NIS (with or without avelumab) in patients with refractory advanced/metastatic solid tumors. The primary safety analysis will be based on the MTD of the VSV-IFNβ-NIS. MTD will be determined by the occurrence of dose-limiting toxicities (DLTs). Safety will be assessed by reported adverse experiences using CTCAE, Version 4.03 and the CRS grading system. The attribution to treatment, time-of-onset, duration of the event, its resolution, and any concomitant medications administered will be recorded. AEs will be analyzed including but not limited to all AEs, SAES, fatal AEs, and laboratory changes.

Safety and tolerability will be assessed by analysis of all relevant parameters including DLTs, adverse events (AEs), laboratory tests, ECGs and vital signs. Count and grade percentage of AE will be provided. Clopper-Pearson 95% confidence interval for the proportion of AE of clinical interest will be estimated using exact method based on binomial distribution. All safety parameters will be presented by dose cohort and overall and by relationship to study drug.

1. A method of treating metastatic colon cancer in a subject in need of treatment thereof, the method comprising administration to the subject of a programmed death-ligand 1 (PD-L1) inhibitor and administration to the subject of a recombinant vesicular stomatitis virus (rVSV) that has been engineered to expresses interferon beta and a sodium/iodine symporter, wherein the PD-L1 inhibitor is an anti-PD-L1 antibody or anti-PD-L1 antibody fragment thereof that specifically binds to PD-L1 and comprises a heavy chain and a light chain, wherein the heavy chain comprises three complementarity determining regions (CDRs) having amino acid sequences of SEQ ID NOs: 1, 2 and 3, respectively, and the light chain comprises three CDRs having amino acid sequences of SEQ ID NOs: 4, 5 and 6, respectively.

2. The method of claim 1, wherein the anti-PD-L1 antibody or anti-PD-L1 antibody fragment thereof mediates antibody-dependent cell-mediated cytotoxicity (ADCC).

3. The method of claim 1, wherein the PD-L1 antibody comprises a heavy chain and a light chain, wherein the heavy chain comprises the amino acid sequence of SEQ ID NOs: 7 or 8 and the light chain comprises an amino acid sequence of SEQ ID NO: 9.

4. The method of claim 1, wherein the PD-L1 inhibitor is avelumab.

5. The method of claim 1, wherein the PD-L1 inhibitor and the rVSV are administered to the subject following disease progression after the subject has received at least one, two, or three lines of cancer therapy.

6. The method of claim 1, wherein the rVSV and the PD-L1 inhibitor are administered sequentially in either order.

7. The method of claim 1, wherein the rVSV is administered prior to the PD-L1 inhibitor.

8. The method of claim 7, comprising (a) under the direction or control of a physician, the subject receiving the rVSV prior to first receipt of the PD-L1 inhibitor; and (b) under the direction or control of a physician, the subject receiving the PD-L1 inhibitor.

9. The method of claim 7, wherein the PD-L1 inhibitor is administered at least one-half, one, two, three, four, five, six, seven, eight, nine ten, 11, 12, 13, 14, 15, 16, 17, 18, 19, 20, 21, 22, 23, 24, 25, 26, 27, 28, 29 or 30 days after the initial administration of the rVSV.

10. The method of claim 1, wherein the PD-L1 inhibitor is administered more than once.

11. The method of claim 1, wherein the rVSV is administered intratumorally.

12. The method of claim 1, wherein the dose of the rVSV is at least about 3×10⁶ 50% tissue culture infective dose (TCID₅₀), 1×10⁷ TCID₅₀, 3×10⁷ TCID₅₀, 1×10⁸ TCID₅₀, 3×10⁸ TCID₅₀, 1×10⁹ TCID₅₀, or 3×10⁹ TCID₅₀ per administration.

13. The method of claim 1, wherein the total dose of the PD-L1 inhibitor administered is at least about 200 mg, 300 mg, 400 mg, 600 mg, 800 mg, 1000 mg, 1200 mg, 1400 mg, 1600 mg, 1800 mg, or 2000 mg per administration.

14. The method of claim 1, wherein the subject is monitored by measurement of immune checkpoints and co-stimulatory molecules on peripheral blood T cell populations.

15. The method of claim 1, wherein the subject is monitored by comparison of pre-treatment and post-treatment immunocyte infiltration.

16. A combination comprising a programmed death-ligand 1 (PD-L1) inhibitor and a recombinant vesicular stomatitis virus (rVSV) that has been engineered to expresses interferon beta and a sodium/iodine symporter, wherein the PD-L1 inhibitor is an anti-PD-L1 antibody or anti-PD-L1 antibody fragment thereof that specifically binds to PD-L1 and comprises a heavy chain and a light chain, wherein the heavy chain comprises three complementarity determining regions (CDRs) having amino acid sequences of SEQ ID NOs: 1, 2 and 3, respectively, and the light chain comprises three CDRs having amino acid sequences of SEQ ID NOs: 4, 5 and 6, respectively.

17. A pharmaceutical composition comprising a programmed death-ligand 1 (PD-L1) inhibitor, a recombinant vesicular stomatitis virus (rVSV) that has been engineered to expresses interferon beta and a sodium/iodine symporter, and a pharmaceutically acceptable carrier, diluent, excipient and/or adjuvant, wherein the PD-L1 inhibitor is an anti-PD-L1 antibody or anti-PD-L1 antibody fragment thereof that specifically binds to PD-L1 and comprises a heavy chain and a light chain, wherein the heavy chain comprises three complementarity determining regions (CDRs) having amino acid sequences of SEQ ID NOs: 1, 2 and 3, and the light chain comprises three CDRs having amino acid sequences of SEQ ID NOs: 4, 5 and 6.

18. (canceled)

19. (canceled)

20. The method of claim 10, wherein, the multiple doses of a PD-L1 inhibitor are administered at least one, two, three, four, five, six, seven, eight, nine ten, 11, 12, 13, 14, 15, 16, 17, 18, 19, 20, 21, 22, 23, 24, 25, 26, 27, 28, 29 or 30 days after the initial administration of the PD-L1 inhibitor.

21. The method of claim 14, wherein the immune checkpoints and co-stimulatory molecules include PD-1, TIM3, and LAG3.


SEQUENCE LISTING (9 sequences)

SEQ ID NO: 1 - protein, 5 aa, Homo sapiens
SYIMM

SEQ ID NO: 2 - protein, 17 aa, Homo sapiens
SIYPSGGITFYADTVKG

SEQ ID NO: 3 - protein, 11 aa, Homo sapiens
IKLGTVTTVDY

SEQ ID NO: 4 - protein, 14 aa, Homo sapiens
TGTSSDVGGYNYVS

SEQ ID NO: 5 - protein, 7 aa, Homo sapiens
DVSNRPS

SEQ ID NO: 6 - protein, 10 aa, Homo sapiens
SSYTSSSTRV

SEQ ID NO: 7 - protein, 450 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 449 aa, Homo sapiens
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 216 aa, Homo sapiens
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS